(19) United States
(12) Patent Application Publication
(10) Pub. No.: US 2021/0309761 A1
(43) Pub. Date: Oct. 7, 2021

(54) METHOD OF INHIBITING THE GROWTH OF LIVER CANCER CELLS, AND METHOD OF TREATING LIVER CANCER

(71) Applicant: THE JIKEI UNIVERSITY, Tokyo (JP)

(72) Inventors: Kohji YAMADA, Tokyo (JP); Ryusuke KIZAWA, Tokyo (JP); Kiyotsugu YOSHIDA, Tokyo (JP)

(73) Assignee: THE JIKEI UNIVERSITY, Tokyo (JP)

(21) Appl. No.: 17/098,790

(22) Filed: Nov. 16, 2020

(30) Foreign Application Priority Data
Sep. 17, 2019 (JP) 2019-168678

(51) Int. Cl. C07K 16/40; A61P 35/00

(52) U.S. Cl. CPC C07K 16/40; A61P 35/00; A61K 2039/505; C07K 2317/76; C07K 2317/55; C07K 2317/24

(57) ABSTRACT

For human liver cancer cell lines which have been found to extracellularly release PKCδ, an analysis of neutralization of the action of PKCδ was carried out using a monoclonal antibody against PKCδ. As a result, it was found that anti-PKCδ antibody or an antigen-binding fragment thereof has a cell growth-inhibiting effect on the liver cancer cell lines.

### TECHNICAL FIELD

The present invention relates to a method of inhibiting the growth of liver cancer cells, and a method of treating liver cancer.

### BACKGROUND ART

Protein kinase C (PKC) is a serine threonine kinase, and is an enzyme which phosphorylates the hydroxyl groups of serine and threonine residues in protein molecules. PKC includes conventional PKC isozymes (α, βI, βII, and γ), which require diacylglycerol (DAG) and calcium ions (CA²⁺) for their activation, and new PKC isozymes (δ, ε, θ, and η), which require only DAG for their activation.

Protein kinase C delta (PKCδ), a new PKC isozyme, is an intracellular signaling kinase of about 78 kilodaltons, and has been well known to be expressed in various cells. However, there has been no report indicating extracellular localization of PKCδ, and whether PKCδ is present also extracellularly has been unclear.

In a past study using a liver cancer cell line, importin al, which is known as a nuclear transport factor, has been found to be present also extracellularly, and to contribute to liver cancer cell growth (Scientific Report 2016; 6: 21410). However, involvement of the function of extracellular PKCδ in cellular functions of various cells such as cancer cells has been unclear. Moreover, there has been no report on the idea of drug discovery targeting extracellularly localized PKCδ.

In general, liver cancer has a poor prognosis and a high recurrence rate. Examples of its curative treatment include liver transplantation and ablation therapy. The curative treatment is applicable only to, for example, the case where the tumor number is not more than three, and where the tumor size is not more than 3 cm. On the other hand, in the case where the tumor number is not less than four, or where the tumor size is larger than 3 cm, the mortality rate is high, leading to death of thirty thousand people in Japan every year.

As a molecular-targeted agent for liver cancer, sorafenib is known. According to a guideline for liver cancer clinical practice, examples of indices for recommendation of application of this molecular-targeted agent include the fact that the tumor number is not less than four. However, even in cases where the molecular-targeted agent is administered in accordance with the guideline, the therapeutic effect by the molecular-targeted agent is often insufficient, so that development of a therapeutic agent that improves the treatment result has been an urgent issue.

### SUMMARY OF THE INVENTION

An aspect of the present invention is to provide a liver cancer cell growth inhibiting method, and a liver cancer treating method.

Regarding localization of the extracellular domain of PKCδ, the present inventors had discovered that it is present in blood of liver cancer patients, and that extracellularly localized PKCδ can be used as a highly accurate marker for liver cancer diagnosis (Japanese Patent Application No. 2018-095674).

Thereafter, in an analysis using recombinant PKCδ, the present inventors discovered that, by allowing PKCδ to act on a liver cancer cell line from outside the cells, PKCδ can have a cell growth-promoting effect on the liver cancer cell line.

Further, for human liver cancer cell lines which had been found to extracellularly release PKCδ, an analysis of neutralization of the action of PKCδ was carried out using a monoclonal antibody against PKCδ. As a result, the present inventors discovered that the monoclonal antibody against PKCδ has a cell growth-inhibiting effect on the liver cancer cell lines.

One aspect of the present invention is to provide a method of inhibiting the growth of liver cancer cells, comprising administering a PKCδ inhibitor such as an anti-PKCδ antibody or an antigen-binding fragment thereof or a chemical inhibitor to a subject in need thereof.

Another aspect of the present invention is to provide a method of treating liver cancer, comprising administering a PKCδ inhibitor such as an anti-PKCδ antibody or an antigen-binding fragment thereof or a chemical inhibitor to a subject in need thereof.

### BRIEF DESCRIPTION OF THE DRAWINGS

FIG. 1 illustrates addition of a recombinant PKCδ (rPKCδ) protein to cells, and its growth-promoting effect on liver cancer cells. A recombinant PKCδ (rPKCδ) protein was added to culture supernatant of a liver cancer cell line (HepG2), and culture was performed for 48 hours. To each well, MIS (3-(4,5-dimethylthiazol-2-yl)-5-(3-carboxymethoxyphenyl)-2-(4-sulfophenyl)-2H-tetrazolium) reagent (Promega) was added, and the absorbance at 450 nm was measured. The absorbance value, which indicates the cell growth rate, is shown as the average from three replicates of the experiment.

FIG. 2 illustrates activation of growth signaling factors, promoted by addition of a recombinant PKCδ protein to cells (photographs). A recombinant PKCδ protein was added to culture supernatant of HepG2 cells that had been subjected to nutrient starvation overnight in a medium supplemented with 0.1% fetal bovine serum (FBS) (Gibco BRL). The cells were cultured for 0, 5, 10, 15, 30, or 60 minutes, and then collected, followed by performing Western blotting. A positive control was provided by addition of a medium supplemented with 10% FBS.

FIG. 3 illustrates immunoblot analysis of PKCδ, AFP, and Importin al in media and lysates of primary cells and several cell lines, following 24 h of incubation. Ponceau-S staining and actin were used as loading controls in the media and lysates, respectively. Primary human hepatocytes were purchased from Lonza (Walkersville, Md., USA) in 2020. Hepatocytes were maintained in HBM basal Medium (Lonza) supplemented with SignalQuots Kit (Lonza).

FIG. 4 illustrates a growth inhibitory action, by treatment with an anti-PKCδ monoclonal antibody, on liver cancer cells. Cells that extracellularly release a large amount of PKCδ (HepG2 or Hep3B) and a cell line (AGS) that hardly shows extracellular release of PKCδ were separately plated on a 96-well plate (3×10³ cells/well). To each well, a mouse IgG of the same isotype (“cont.” in the figure) or a mouse anti-PKCδ monoclonal antibody (“clone 14” in the figure) (BD, Clone 14) was added at a concentration of 1 μg/ml, and culture was performed for 48 hours. Thereafter, MTS reagent was added to each well, and the absorbance at 450 nm was measured. The absorbance value, which indicates the cell growth rate, shown is the average from three replicates of the experiment.

FIG. 5 illustrates an inhibitory action, by treatment with an anti-PKCδ monoclonal antibody, on activation of growth signaling factors (photographs). Immunoblot analysis of phospho-IGF1R (Y1131), phospho-ERK1/2, phospho-STAT3 (Y705), total IGF1R, total ERK1/2, total STAT3, and Actin (loading control) in HepG2 and Hep3B liver cancer cells treated with isotype control or anti-PKCδ monoclonal antibody (100 ng/mL) (BD, Clone 14) and then cultured for 5, 10, or 60 minutes, followed by collecting the cells and performing Western blotting. The data represent four experiments.

FIG. 6 illustrates an inhibitory effect, by treatment with an anti-PKCδ monoclonal antibody, on spheroid formation (photographs). HepG2 cells were cultured on a low attachment plate for 5 days in the presence of a mouse IgG of the same isotype (“control” in the figure) or a mouse anti-PKCδ monoclonal antibody (“α-PKCδ mAb” in the figure) (BD, Clone 14), and spheroid formation was observed.

FIG. 7 illustrates a decrease in the growth ability of spheroid-forming cells, caused by treatment with an anti-PKCδ monoclonal antibody. HepG2 cells were cultured on a low attachment plate for 5 days in the presence of a mouse IgG of the same isotype (“0 ng/ml” in the figure) or a mouse anti-PKCδ monoclonal antibody (“10”, “100”, or “1000 ng/ml” in the figure) (BD, Clone 14). Thereafter, MTS reagent was added to each well, and the absorbance at 450 nm was measured. The absorbance value, which indicates the cell growth rate, shown is the average from three replicates of the experiment.

FIG. 8 illustrates decrease in the tumor volume, by treatment with an anti-PKCδ monoclonal antibody. Hep3B cells were inoculated s.c. into nude mice. Mice were treated with intratumoral injection of isotype control (n=6) or anti-PKCδmonoclonal antibody (n=6) (0.5 mg/Kg) at thrice per week. Tumor volume was monitored.

FIG. 9 illustrates decrease in the tumor size (photograph) and weight, by treatment with an anti-PKCδ monoclonal antibody. Macroscopic images and tumor weight of Hep3B (n=6 per group) tumors treated with isotype control or anti-PKCδ monoclonal antibody at 19 days after cell injection. Error bars represent mean±SD. *p<0.001, using Student's t-test.

FIG. 10 illustrates an inhibitory action, by treatment with an anti-PKCδ monoclonal antibody, on activation of growth signaling factors (photographs). Hep3B tumor tissues were stained with H&E, anti-Ki67 antibody, anti-phospho-IGF1R antibody, and anti-phosho-ERK1/2 antibody. Hep3B tumor tissue was obtained by removing the subcutaneously transplanted tumor cell mass and then sectioning the tissue. Scale bars, 20 μm.

FIG. 11 illustrates decrease in the tumor size (photograph) and weight, by treatment with an anti-PKCδ monoclonal antibody. Macroscopic images and tumor weight of HuH7 (n=3 per group) tumors treated with isotype control or anti-PKCδ monoclonal antibody at 17 days after cell injection. Error bars represent mean±SD. *p<0.05, using Student's t-test.

### MODES FOR CARRYING OUT THE INVENTION

The present invention provides a method of inhibiting the growth of liver cancer cells, comprising administering a PKCδ inhibitor such as an anti-PKCδ antibody or an antigen-binding fragment thereof or a chemical inhibitor to a subject in need thereof.

The anti-PKCδ antibody is not limited as long as it is capable of binding to PKCδ protein, and examples thereof include antibodies having a neutralizing action, antibodies having CDC (complement-dependent cytotoxicity) activity, and antibodies having ADCC (antibody-dependent cell-mediated cytotoxicity) activity. The anti-PKCδ antibody is preferably an antibody that extracellularly binds to PKCδ protein to neutralize the action of the PKCδ protein. The anti-PKCδ antibody may also be an antibody that binds to PKCδ protein expressed on the cell membrane of liver cancer cells, to neutralize the action of the PKCδ protein.

When necessary, for enhancing the above-described action or activity of the antibody, or for giving another action or activity to the antibody, an arbitrary compound may be bound to an arbitrary position on the antibody. The compound may be a drug.

The action of PKCδ protein herein means, for example, an action of PKCδ protein on liver cancer cells. Examples of the action include those in cases where PKCδ protein acts on a sugar chain or a protein such as a receptor on the surface of liver cancer cells to activate a signal transducer involved in the cell growth, to thereby promote the cell growth of the liver cancer cells.

PKCδ is a protein expressed in various cells, and localized intracellularly in cells other than liver cancer cells. However, liver cancer cells extracellularly release part of PKCδ.

The epitope of the anti-PKCδ antibody used in the present invention is not limited as long as the antibody recognizes PKCδ which is present extracellularly. Examples of the antibody include those which recognize an epitope included in the amino acid sequence represented by amino acid positions 114 to 289 of SEQ ID NO:1. The antibody may also be an antibody that recognizes an epitope included in an amino acid sequence having a sequence identity of not less than 95%, preferably not less than 98% to the amino acid sequence represented by amino acid positions 114 to 289 of SEQ ID NO: I.

The anti-PKCδ antibody may be either a polyclonal antibody or monoclonal antibody. From the viewpoint of achieving a stable therapeutic effect, the antibody is preferably a monoclonal antibody.

Further, for use in treatment of a human, the anti-PKCδ antibody is preferably a chimeric antibody, humanized antibody, or fully human antibody from the viewpoint of decreasing antigenicity.

The antibody used may be a commercially available antibody, or may be an antibody prepared by a method known to those skilled in the art.

In cases of a monoclonal antibody, examples of the method of preparing the antibody include a method in which an animal such as a mouse is immunized using PKCδ as an antigen, and then cells producing an antibody against PKCδ antigen protein are collected, followed by fusing the collected cells with myeloma cells of the same or different species, selecting a hybridoma cell producing an anti-PKCδ monoclonal antibody, and then obtaining the antibody from culture supernatant of the hybridoma cell.

Further, by modification of the hybridoma cell, a chimeric antibody or a humanized antibody can be obtained. More specifically, for example, an antibody of interest can be obtained by a method known to those skilled in the art based on a genetic recombination technique, wherein a portion encoding the Pc region in a gene extracted from the hybridoma cell is replaced with a gene encoding a human Fc region.

Regarding the fully human antibody of the anti-PKCδ antibody, a genetically modified mouse or the like capable of producing human antibody may be immunized using PKCδ as an antigen, and anti-PKCδ-antibody-producing cells obtained from the genetically modified mouse may be collected, followed by fusing the collected cells with myeloma cells, and selecting a hybridoma cell producing an anti-PKCδ antibody. The fully human antibody can be obtained from culture supernatant of the hybridoma cell.

Alternatively, the fully human antibody of the anti-PKCδ antibody can be prepared by using the phage display method, which is a method known to those skilled in the art.

The antigen-binding fragment is not limited as long as it is capable of binding to the antigen protein PKCδ, and examples thereof include Fab, Fab′, F(ab′)₂, scFab, scFv, diabodies, triabodies, and minibodies. Any of these antigen-binding fragments may be produced by using gene modification techniques known to those skilled in the art.

The PKCδ inhibitor may be a chemical compound having a PKCδ-inhibitory activity (chemical inhibitor). Examples of such compounds include, but are not limited to, rottlerin (CAS 82-08-6), Delcasertib (KAI-9803), bisindolylmaleimide T, bisindolylmaleimide II, bisindolylmaleimide III, bisindolylmaleimide IV, calphostin C, chelerythrine chloride, ellagic acid, Go 7874, Go 6983, H-7, Iso-H-7, hypericin, K-252a, K-252b, K-252c, melittin, NGIC-I, phloretin, staurosporine, polymyxin 13 sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, safmgol, sangivamycin, D-erythro-sphingosine, or derivative or prodrug thereof, and combinations thereof. The compounds disclosed in U.S. Pat. No. 9,844,534B2, U.S. Pat. No. 9,572,793B2, and U.S. Pat. No. 9,364,460B2 can also be used as PKCδ inhibitor.

Examples of the liver cancer include, but are not limited to, hepatocellular carcinoma, cholangiocellular carcinoma, mixed liver cancer, metastatic liver cancer, hepatoblastoma, and fibrolamellar hepatocellular carcinoma (fibrolamellar HCC). The term “liver cancer” is not meant to limit the diseased site in the liver, the disease stage, or the like, and includes any of diseased sites, disease stages, and the like.

Another aspect of the present invention is a method of treating liver cancer comprising administering an effective amount of a PKC inhibitor such as an anti-PKCδ antibody or an antigen-binding fragment thereof or a chemical inhibitor to a subject in need thereof.

The PKCδ inhibitor such as an anti-PKCδ antibody or an antigen-binding fragment thereof or a chemical inhibitor may be administered to a subject as it is, or may be administered to a subject as a liver cancer therapeutic agent prepared by mixing with another effective component and/or a pharmaceutically acceptable carrier.

The content of the PKCδ inhibitor such as an anti-PKCδ antibody or an antigen-binding fragment thereof or a chemical inhibitor in the liver cancer therapeutic agent is not limited as long as growth inhibition of liver cancer cells is possible therewith. The content is preferably 1 ng/ml to 10 μg/ml.

The liver cancer therapeutic agent comprising a PKC inhibitor such as an anti-PKCδ antibody or an antigen-binding fragment thereof or a chemical inhibitor may be formulated into an arbitrary dosage form. Examples of the dosage form include solids such as powder or tablet, liquids, suspensions, and injection solutions. The agent is preferably an injection solution.

The mode of administration is not limited, and is preferably oral administration or topical administration to the affected area or its vicinity by injection or the like, or intravenous injection.

Examples of other effective components include immunostimulating substances such as cytokines, and chemotherapeutic agents. These other effective components may be used as appropriate in appropriate amounts.

Examples of the pharmaceutically acceptable carrier include solvents, distilled water, physiological saline, diluents, surfactants, stabilizers, solubilizers, suspending agents, emulsifiers, buffers, and preservatives. In addition, when necessary, additives such as antiseptics, antioxidants, coloring agents, adsorbents, and wetting agents may be used. These carriers may be used as appropriate in appropriate amounts.

The subject to which the liver cancer therapeutic agent is administered is a mammal, preferably a human.

The dose of the liver cancer therapeutic agent is not limited as long as the effective component, a PKCδ inhibitor such as anti-PKCδ antibody or an antigen-binding fragment thereof or a chemical inhibitor, is capable of inhibiting growth of liver cancer cells, to produce a therapeutic effect against liver cancer. The dose may be appropriately adjusted depending on the age, sex, body weight, symptoms, therapeutic effect, area of the treatment site, administration method, and the like. For example, in cases where an average human having a body weight of about 60 kg is to be treated, the dose is preferably about 0.01 mg to 5000 mg, more preferably 0.1 mg to 500 mg per day. The total daily dose may be achieved with either a single dose or divided doses.

Regarding the therapeutic effect, when in vivo analysis is carried out, the liver cancer therapeutic agent can be judged to have a therapeutic effect in cases, for example, where treatment with the liver cancer therapeutic agent results in inhibition of growth of liver cancer cells, where liver cancer cells decrease, and/or where the size of the liver cancer decreases, as confirmed based on comparison with the state before the treatment with the liver cancer therapeutic agent, or based on comparison with a control that is not subjected to the treatment with the liver cancer therapeutic agent. In these cases, the analysis method is not limited, and may be a method known to those skilled in the art.

When cell biological analysis is carried out, the therapeutic effect of the therapeutic agent in vivo can be predicted by comparison between samples after treatment with the therapeutic agent and samples before treatment with the therapeutic agent. Examples of the cell biological analysis include, but are not not limited to, a cell growth assay, a cell cluster (spheroid) formation assay, and Western blotting. The analysis may be carried out by a method known to those skilled in the art.

### EXAMPLES

Examples are described for the purpose of disclosure, and not meant to limit the scope of the present invention.

As the methods of molecular biology, cell biology, and immunology which are mentioned in the present disclosure and Examples, but which are not clearly described, conventional methods well known to those skilled in the art are used. Such techniques are sufficiently described in literatures such as “Methods in Molecular Biology”, published by Humana Press; “Molecular Cloning: A Laboratory Manual, second edition” (Sambrook et al., 1989), published by Cold Spring Harbor Press; “Cell Biology: A Laboratory Notebook” (J. E. Cellis, ed., 1998), published by Academic Press; “Current Protocols in Molecular Biology” (F. M. Ausubel et al., eds., 1987); “Short Protocols in Molecular Biology” (Wiley and Sons, 1999); “Introduction to Cell and Tissue Culture” (J. P. Mather and P. E. Roberts, 1998), published by Plenum Press; “Animal Cell Culture” (R. I. Freshney ed., 1987); “Cell and Tissue Culture: Laboratory Procedures” (A. Doyle, J. B. Griffiths, and D. G. Newell, eds., 1993-1998), J. Wiley and Sons; “Handbook of Experimental Immunology” (F. M. Ausubel et al., eds, 1987); “Current Protocols in Immunology” (J. E. Coligan et al., eds., 1991); and “Methods in Enzymology” (Academic Press).

### <Materials and Methods>

### Cell Culture

Liver cancer cell lines (HepG2 and Hep3B) and a gastric cancer cell line AGS were cultured in DMEM or RPMI1640 medium (Nacalai) supplemented with 10% fetal bovine serum (FBS) (Gibco BRL), penicillin (100 units/nil), and streptomycin (100 μg/ml) (Nacalai). All cell lines were obtained from the cell bank (JCRB) in the National Institutes of Biomedical Innovation, Health and Nutrition, and grown under humidified conditions at 5% CO₂ at 37° C.

### SDS-PAGE and Western Blotting

A whole-cell lysate was prepared as described elsewhere (Scientific Report 2016; 6: 21410). A protein sample was developed by polyacrylamide gel electrophoresis (SOS-PAGE), and then transferred to a nitrocellulose membrane. Thereafter, specific antigens were reacted with their corresponding antibodies, and then reaction with secondary IgG (Santa Cruz) conjugated with horseradish peroxidase (HRP) was carried out. After washing the nitrocellulose membrane, visualization by the enhanced chemiluminescence method (ECL method) was carried out.

### Spheroid Formation

On a 6-well plate with an ultra-low attachment surface (Corning), 2×10³ HepG2 cells were plated. As a medium, DMEM-Ham's F-12 (Nacalai) supplemented with EGF (recombinant human epidermal growth factor), FGF (recombinant human fibroblast growth factor), recombinant human insulin, and B27 serum-free supplement (Thermo) was used. Five days later, spheroid formation was investigated using a phase contrast microscope.

### Cell Growth Assay

Cells were grown in a culture liquid in a total volume of 100 μl (3×10³ cells per well) in the presence of a mouse anti-PKCδ monoclonal antibody (1 μg/ml) (BD, Clone 14) or an isotype mouse control IgG (1 μg/ml) (Santa Cruz). Forty-eight hours later, MTS reagent (Promega) was added to each well, followed by 30 minutes of incubation. A water-soluble formazan dye generated by bioreduction was measured using a microplate reader. All samples were tested in four replicated systems, and the average for four replicated wells was used as each measurement value.

### Example 1: Extracellularly Localized PKCδ Functions to Promote Cell Growth

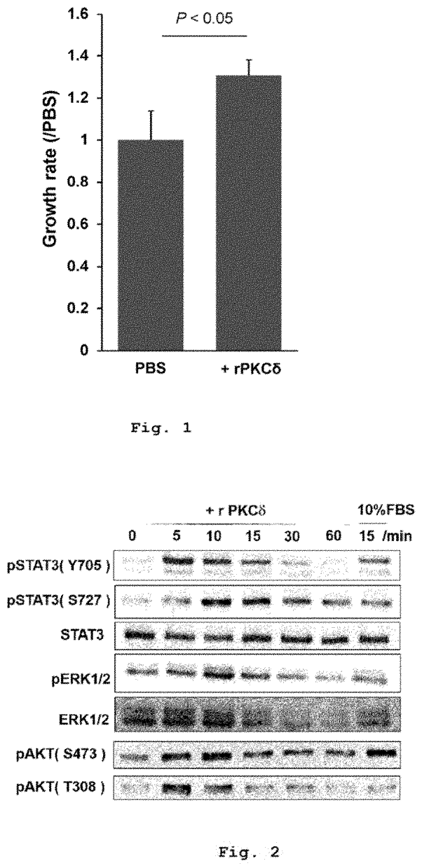

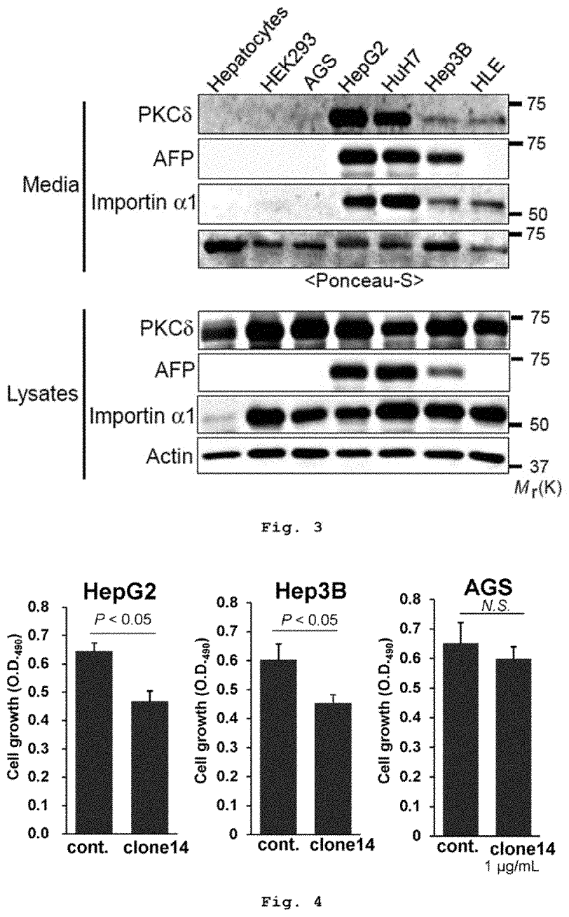

Protein kinase C delta (PKCδ) is well known as an intracellular signaling kinase of about 78 kilodaltons. However, its extracellular function has not been known. Some intracellular proteins detected extracellularly are known to be localized on the cell membrane (Scientific Report 2016; 6: 21410 and JP 2014-6129 A). The present inventors added a PKCδ recombinant protein to culture supernatant of a liver cancer cell line, and investigated the effect of extracellularly localized PKCδ recombinant protein on the cell growth of a liver cancer cell line. As a result, significant promotion of the cell growth was found for cells treated with the PKCδ recombinant protein (FIG. 1). Further, the effect of extracellularly localized PKCδ on the intracellular signaling system was investigated using a phosphorylation protein array. As a result, promoted phosphorylation of STAT3 and ERK1/2 was found for cells treated with the PKCδ recombinant protein (data not shown). In order to verify these results, the phosphorylation state after the treatment with the PKCδ recombinant protein was investigated with time using Western blotting. As a result, enhanced phosphorylation of STAT3 and ERK1/2 was found to have occurred 5 minutes to 10 minutes after the treatment (FIG. 2). In particular, since ERK1/2 is an intracellular signaling factor directly involved in the cell growth, it was suggested that extracellularly localized PKCδ contributes to the cell growth of liver cancer cells.

In order to show the cell-type specificity of the extracellular PKCδ, several types of cell lines that endogenously express PKCδ were examined: four liver cancer lines (IIepG2, IIep3B, IIuII7, IILE), a gastric cancer line (AGS) and human embryonic kidney line (HEK293), and primary human normal hepatocytes (FIG. 3). Although intracellular PKCδ levels at both mRNA and protein expression were comparable among all tested cell lines, a large amount of PKCδ was detected in the conditioned medium (CM) of all of liver cancer cell lines, whereas the AGS and HEK293 cell lines showed no or low PKCδ levels in their CM. In addition, it was found that no PKCδ levels was detectable in the CM of hepatocytes.

### Example 2: Anti-PKCδ Monoclonal Antibody Inhibits Cell Growth of Liver Cancer Cell Line

In order to investigate whether extracellularly localized PKCδ is involved in growth of liver cancer cells, a cell growth assay was carried out using an isotype mouse control IgG and a mouse anti-PKCδ monoclonal antibody (BD, Clone 14). As a result, a significant cell growth-inhibiting effect could be found for liver cancer cell lines that extracellularly release a large amount of PKCδ (HepG2 and Hep3B) (FIG. 4). On the other hand, AGS, which is a gastric cancer cell line that hardly extracellularly releases PKCδ, showed no significant cell growth-inhibiting effect by the treatment with the anti-PKCδ monoclonal antibody. Subsequently, Western blotting was carried out to investigate activation of cell growth signaling factors.

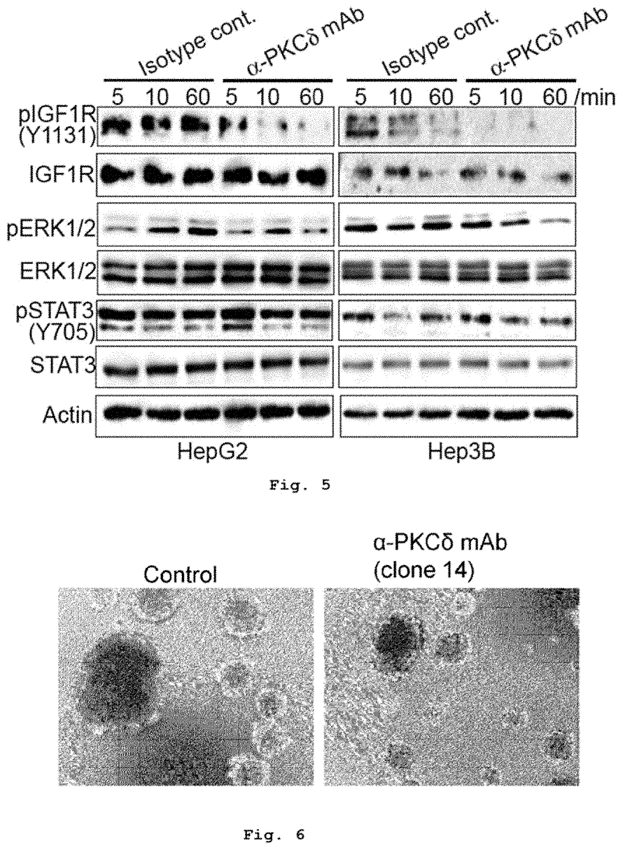

It was also found that the anti-PKCδ monoclonal antibody treatment reduced the phosphorylation level of IGF1R and ERK1/2, but not STAT3 in PKCδ-positive liver cancer cell lines, suggesting that activation of ERK1/2 by extracellular PKCδ plays a critical role in liver cancer cell proliferation (FIG. 5). Thus, it was shown that extracellularly localized PKCδ contributes to the growth mechanism of liver cancer cells, and that targeting of extracellularly localized PKCδ with an antibody or the like can be utilized for treatment of liver cancer.

### Example 3: Anti-PKCδ Monoclonal Antibody Inhibits Spheroid-Forming Ability of Liver Cancer Cells

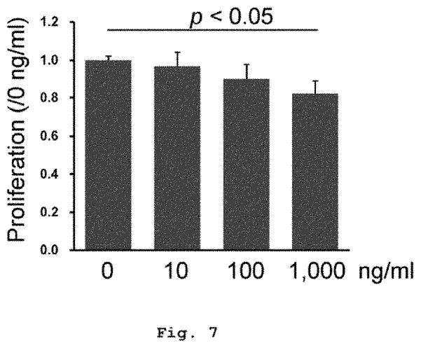

In order to investigate whether extracellularly localized PKCδ is involved in the tumorigenicity of liver cancer cells, a spheroid formation experiment was carried out using HepG2 cells. Compared to the group treated with the isotype mouse control IgG, the group treated with the mouse anti-PKC& monoclonal antibody showed formation of smaller spheroids, indicating that the latter group tends to have a weaker spheroid-forming ability (FIG. 6). Further, in a cell growth assay, the group treated with the mouse anti-PKCδ monoclonal antibody showed a significant decrease in the cell growth rate compared to the group treated with the isotype mouse control IgG (FIG. 7). Thus, it was suggested that extracellularly localized PKCδ may be directly involved in the tumorigenicity.

### Example 4: Anti-PKCδ Monoclonal Antibody Inhibits the Growth of Liver Cancer Cells In Vivo

All animal studies were approved by the Animal Care and Use Committee of Jikei University School of Medicine (the ethical approval number: 28-16) and conducted in accordance with the guidelines for animal experimentation established at Jikei University School of Medicine (Tokyo, Japan). Male nude mice (6-7 weeks old) were obtained from CLEA (Tokyo, Japan). The animals were maintained in a pathogen-free animal facility of Jikei University. Mice were randomized into indicated groups. Cells in 100 mL of Matrigel were implanted subcutaneously in the back flank of the mice. Mice were injected intratumorally with isotype control IgG or anti-PKCδ monoclonal antibody (0.5 mg/Kg per injection). Antibody administration was performed 3 times per week from 2 days after cell administration, for a total of 7-8 times. Tumor size was determined by caliper measurement of the largest (x) and smallest (y) perpendicular diameters and was calculated according to the formula V=π/6×xy².

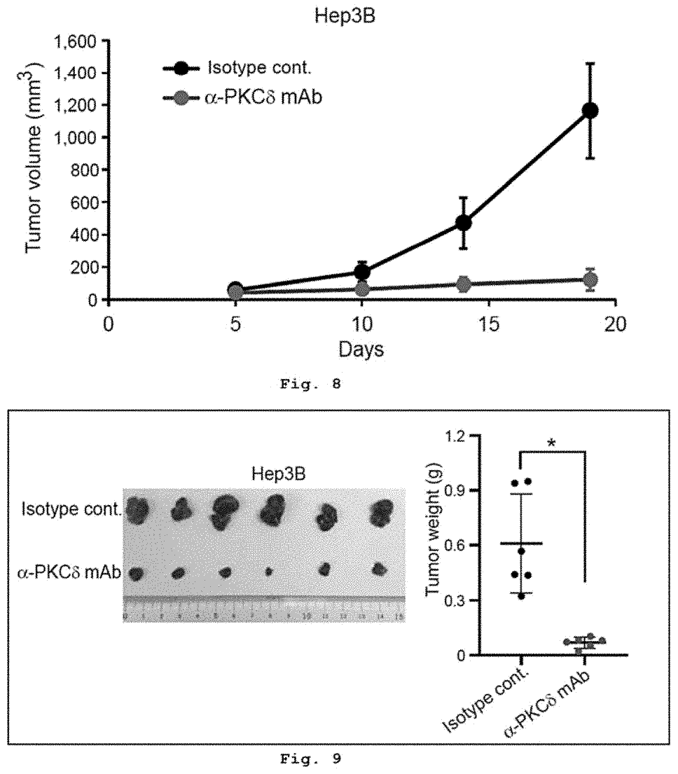

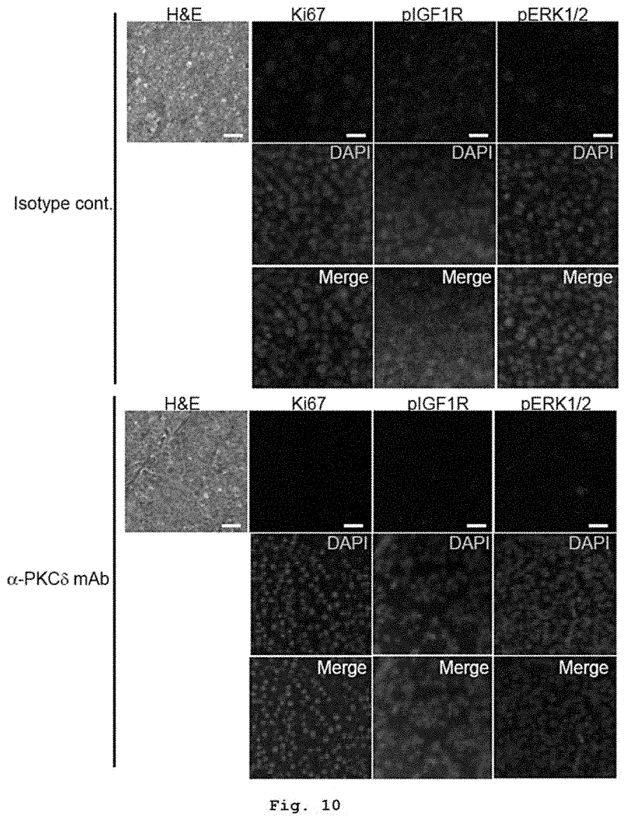

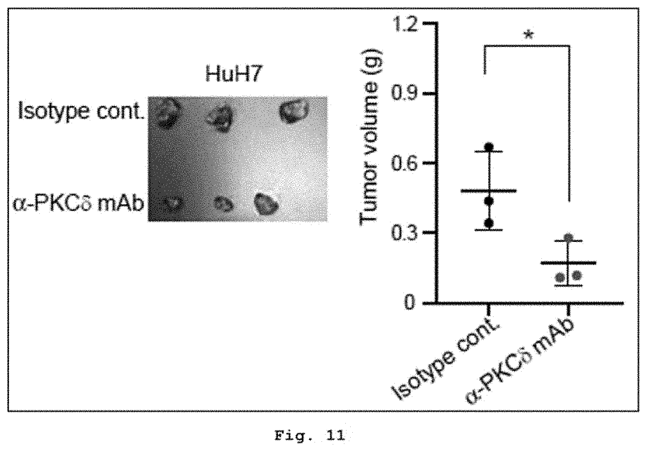

When a xenograft tumor model of liver cancer cells (Hep3B and HuH7) was generated, the tumor volume (FIG. 8), size and weight (FIGS. 9 and 11) were apparently diminished in xenografted mice administrated with the anti-PKCδ monoclonal antibody, compared to that in the mice injected with isotype control IgG. Immunostaining study showed that the anti-PKCδ monoclonal antibody treatment markedly diminished the number of Ki67-positive cells in tumor tissues (FIG. 10). Similarly, the anti-PKCδ monoclonal antibody treatment attenuated phosphorylation levels of IGF1R and ERK1/2 in the tumor tissues (FIG. 10). Taken together, these results suggest that extracellular PKCδ may contribute to tumor growth in liver cancer

These results suggest that extracellularly localized PKCδ is involved in the growth mechanism of liver cancer cells, and that the growth of liver cancer cells can be inhibited by neutralizing extracellularly localized PKC with a specific antibody.

A chemical inhibitor for PKCδ such as rottlerin and Delcasertib is added in a medium and liver cancer cells such as HepG2, Hep3B, HuH7, or HLE are cultured for about 1 day to 3 days. Thereby, the effects of such chemical PKCδ inhibitors on the growth of liver cancer cells can be evaluated.

A chemical inhibitor for PKCδ such as rottlerin and Delcasertib is orally or intravenously administrated to an animal model for liver cancer for about one week to one month. Thereby, the effects of such chemical PKCδ inhibitors on the progression of liver cancer cells can be evaluated.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method of inhibiting the growth of liver cancer cells, comprising administering a PKCδ inhibitor to a subject in need thereof.

2. The method according to claim 1, wherein the PKCδ inhibitor is a chemical compound having a PKCδ-inhibitory activity.

3. The method according to claim 1, wherein the PKCδ inhibitor is an anti-PKCδ antibody or an antigen-binding fragment thereof.

4. The method according to claim 3, wherein the antibody is an antibody that neutralizes an action of extracellular PKCδ.

5. The method according to claim 3, wherein the antibody is an antibody which recognizes an epitope in the amino acid sequence represented by amino acids 114 to 289 of SEQ ID NO:1.

6. The method according to claim 3, wherein the antibody is an antibody which recognizes an epitope in an amino acid sequence having a sequence identity of not less than 95% to the amino acid sequence represented by amino acids 114 to 289 of SEQ ID NO: 1.

7. The method according to claim 3, wherein the antibody is a chimeric antibody, humanized antibody, or fully human antibody.

8. The method according to claim 3, wherein the antigen-binding fragment is Fab, Fab′, F(ab′)₂, scFab, scFv, diabody, triabody, or minibody.

9. A method of treating liver cancer, comprising administering a PKCδ inhibitor to a subject in need thereof.

10. The method according to claim 9, wherein the PKCδ inhibitor is a chemical compound having a PKCδ-inhibitory activity.

11. The method according to claim 9, wherein the PKC inhibitor is an anti-PKCδ antibody or an antigen-binding fragment thereof.

12. The method according to claim 11, wherein the antibody is an antibody that neutralizes an action of extracellular PKCδ.

13. The method according to claim 11, wherein the antibody is an antibody which recognizes an epitope in the amino acid sequence represented by amino acids 114 to 289 of SEQ ID NO: 1.

14. The method according to claim 11, wherein the antibody is an antibody which recognizes an epitope in an amino acid sequence having a sequence identity of not less than 95% to the amino acid sequence represented by amino acids 114 to 289 of SEQ ID NO: 1.

15. The method according to claim 11, wherein the antibody is a chimeric antibody, humanized antibody, or fully human antibody.

16. The method according to claim 11, wherein the antigen-binding fragment is Fab, Fab′, F(ab′)₂, scFab, scFv, diabody, triabody, or minibody.


SEQUENCE LISTING (1 sequence)

SEQ ID NO: 1 - protein, 676 aa, Homo sapiens
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED